(19) United States
(12) Patent Application Publication
(10) Pub. No.: US 2002/0192799 A1
(43) Pub. Date: Dec. 19, 2002

(54) Drug discovery methods

(75) Inventors: Stanley J. Watowich, Houston, TX (US); Scott C. Weaver, Galveston, TX (US); Robert A. Davey, Galveston, TX (US)

Correspondence Address: MUETING, RAASCH & GEBHARDT, P.A., P.O. BOX 581415, MINNEAPOLIS, MN, 55458, US

(73) Assignee: Board of Regents, The University of Texas System, Austin, TX (US)

(21) Appl. No.: 09/981,286

(22) Filed: Oct. 15, 2001

Related U.S. Application Data
(60) Provisional application No. 60/240,187, filed on Oct. 13, 2000.

(51) Int. Cl. C12N 9/64; C12P 21/02; C12N 5/06; C12N 9/20

(52) U.S. Cl. 435/226; 435/69.1; 435/320.1; 435/325; 435/198

(57) ABSTRACT

The present invention provides collections of polypeptides, where each polypeptide includes a region Xaaₙ, wherein n is from about 5 to about 21, and each Xaa is independently a random amino acid. Polynucleotides encoding the polypeptides, are also provided, as are methods for identifying a polypeptide within a collection that prevents cell death after exposure to a pathogen or a toxin, and methods for identifying a polypeptide within a collection that binds a pathogen, a toxin, a polypeptide, or a polynucleotide. The present invention also provides methods for crystallizing a polypeptide.

### CONTINUING APPLICATION DATA

This application claims the benefit of U.S. Provisional Application Serial No. 60/240,187, filed Oct. 13, 2000, which is incorporated by reference herein.

### GOVERNMENT FUNDING

The present invention was made with government support under Grant No. DAAD19-01-1-0361, awarded by Defense Advanced Research Projects Agency. The Government has certain rights in this invention.

### BACKGROUND

Pathogenic viruses, bacteria and bacterial toxins, fungi, and parasites are the cause of serious health problems for both humans and animals, and potential agents for biological and agricultural warfare and terrorism (BWT). Effective vaccines and therapeutic drugs are not available for the vast majority of these pathogens. At present, the medical and public health response to a pathogenic infections relies on minimizing transmission and providing palliative care, and treatment when appropriate with anti-bacterial or anti-fungal agents. Developing fast-acting therapeutic compounds to inhibit the replication of many dissimilar pathogens is essential to improving human and animal health, and the ability to respond to a BWT incident.

Previous approaches to discovering and developing antimicrobial countermeasure have relied on (1) screening chemical compounds with little or no a priori knowledge of their potential action or targets, and (2) screening combinatorial libraries of random amino acid or nucleotide sequences for compounds that bind tightly to a favored target biomolecule. These approaches are extremely costly and time-consuming, but rarely successful. Moreover, these approaches are generally specific to only one pathogen or a closely related group of pathogens.

Several complementary studies have recently shown that small in vivo combinatorial libraries can be used to select peptides that produce a phenotypic response. These responses included inhibition of the yeast spindle checkpoint and increased mating pheromone response (Norman et al., Science, 285, 591-595 (1999)), and binding to the REV responsive element of the human immunodeficiency virus RNA (Tan and Frankel, Proc. Natl. Acad. Sci. U.S.A., 95, 4247-4252 (1998)).

### SUMMARY OF THE INVENTION

The present invention represents an advance in the art of discovering drugs that can be used to prevent morbidity and mortality associated with pathogens and toxins, including, for instance, highly lethal agents that could be used in biological warfare. Despite extensive effort, effective countermeasures against agents that can be used in biological warfare threats do not yet exist. Current approaches to developing antiviral compounds include screening natural products or chemicals in vivo, combinatorial organic synthesis, high throughput in vitro screening, and X-ray analysis of ligand-protein co-crystals. These conventional approaches are extremely costly and time-consuming, but rarely successful. The present invention provides a technology which rapidly generates medical countermeasures and identifies validated molecular targets essential for pathogen replication. These countermeasures are designed for use before or after exposure to pathogens and toxins to prevent disease.

The present invention is directed to a collection of polypeptides that includes at least two polypeptides. Each polypeptide includes a fragment of SEQ ID NO:1 beginning at any amino acid from about 119 to about 124 and ending at any amino acid from about 258 to about 275. At least two consecutive amino acids within the regions of amino acids 129-137, or amino acids 182-189, or amino acids 257-264 as depicted at SEQ ID NO:1 are replaced by an amino acid sequence that includes Xaaₙ, wherein n is from about 5 to about 21, and each Xaa is independently a random amino acid. Two examples of polypeptides that are members of one collection are SEQ ID NO:33 and SEQ ID NO:34. Each member of the collection may further include a cell-permeant region fused to the amino terminal end of the polypeptide. Preferably, the cell-permeant region includes an amino acid sequence YGRKKRRQRRR (SEQ ID NO:2), RQIKIWFQNRRMKWKK (SEQ ID NO:3), RQIKIWFPNRRMKWKK (SEQ ID NO:4), or RQPKIWFPNRRPKWKK (SEQ ID NO:5). The invention is further directed to a cell that includes a member of the collection of polypeptides, and a population of cells that includes two or more cells, wherein each member of the population includes one polypeptide of the collection of polypeptides.

The invention also provides a polypeptide selected from the group consisting of an amino acid sequence SEQ ID NO:2 fused to an amino terminal end of a fragment of SEQ ID NO:1 beginning at any amino acid from about 119 to about 124 and ending at any amino acid from about 258 to about 275, wherein at least two consecutive amino acids within the regions of amino acids 129-137, or amino acids 182-189, or amino acids 257-264 as depicted at SEQ ID NO:1 are replaced by an amino acid sequence including Xaaₙ, wherein n is from about 5 to about 21, and each Xaa is independently a random amino acid. The polypeptide may further include a cell-permeant region fused to the amino terminal end of the polypeptide. The invention is further directed to a cell that includes a member of the collection of polypeptides.

Further provided by the invention is a collection of polynucleotides including at least two polynucleotides. Each polynucleotide includes a coding sequence encoding a polypeptide that includes a fragment of SEQ ID NO:1 beginning at any amino acid from about 119 to about 124 and ending at any amino acid from about 262 to about 275, wherein at least two consecutive amino acids within the regions of amino acids 129-137, or amino acids 182-189, or amino acids 257-264 as depicted at SEQ ID NO:1 are replaced by an amino acid sequence including Xaaₙ, wherein n is from about 5 to about 21, and each Xaa is independently a random amino acid. The polypeptide may further include a cell-permeant region fused to the amino terminal end of the polypeptide. The nucleotide sequence of the coding sequence encoding the Xaa may consists of a nucleotide sequence NNKₘ, wherein N is independently a random nucleotide, K is independently a guanine or a thymine, and wherein o is from about 5 to about 21. The polynucleotide may be present in a vector, for instance, a retrovirus vector. The invention is further directed to a cell that includes a member of the collection of polynucleotides, and a population of cells that includes two or more cells, wherein each member of the population includes one polynucleotide of the collection of polynucleotides.

The present invention provides a method for crystallizing a polypeptide that includes an amino acid sequence SEQ ID NO:1. The method includes preparing purified polypeptide that includes an amino acid sequence SEQ ID NO:1 at a concentration of about 3 mg/ml to about 20 mg/ml, and crystallizing the polypeptide from a solution containing about 20% by weight to about 28% by weight polyethylene glycol, about 0.05 M to about 0.2 M ammonium sulfate, and about 1 mM to about 20 mM urea, wherein the solution is buffered to a pH of about 6 to about 8. Another method for crystallizing a polypeptide that includes an amino acid sequence SEQ ID NO:1 includes preparing purified polypeptide that includes an amino acid sequence SEQ ID NO:1 at a concentration of about 3 mg/ml to about 20 mg/ml, and crystallizing the polypeptide from a solution that contains about 15% by weight to about 25% by weight polyethylene glycol 4000, and about 0.05 M to about 0.4 M MgCl2, wherein the solution is buffered to a pH of about 6 to about 8.

The invention is further directed to a crystal of a polypeptide that includes an amino acid sequence SEQ ID NO:1. Preferably, the crystal has the space group symmetry P2₁2₁2₁. Preferably, the crystal includes a unit cell having dimensions of a, b, and c; wherein a is about 69.3 Å to about 72.0 Å, b is about 75.2 Å to about 76.0 Å, and c is about 90.1 Å to about 94.7 Å; and wherein α=β=γ=about 90°.

Also provided by the invention is a method for identifying a polypeptide within a collection that prevents cell death after exposure to a pathogen or a toxin. The method includes providing a cell that contains a polypeptide that is a member of a collection of polypeptides including at least two polypeptides. Each polypeptide includes a fragment of SEQ ID NO:1 beginning at any amino acid from about 119 to about 124 and ending at any amino acid from about 258 to about 275, wherein at least two consecutive amino acids within the regions of amino acids 129-137, or amino acids 182-189, or amino acids 257-264 as depicted at SEQ ID NO:1 are replaced by an amino acid sequence including Xaaₙ, wherein n is from about 5 to about 21, and each Xaa is independently a random amino acid. The cell is exposed to a pathogen or a toxin, and whether the polypeptide prevents cell death is determined by incubating the cell under conditions such that the pathogen or the toxin kills a cell that does not include a polypeptide that prevents cell death after exposure to a pathogen or a toxin. The presence of a cell that proliferates indicates the polypeptide prevents cell death after exposure to a pathogen or a toxin. The pathogen may be, for instance, a virus or a microbe. Examples of microbes include a bacterium, a rickettsia, and a fungus. Examples of toxins include a biological toxin or a chemical toxin.

The invention provides a method for identifying a polypeptide within a collection that binds a pathogen, a toxin, a polypeptide, or a polynucleotide. The method includes providing a cell that includes a polypeptide that is a member of a collection of polypeptides including at least two polypeptides. Each polypeptide includes a fragment of SEQ ID NO:1 beginning at any amino acid from about 119 to about 124 and ending at any amino acid from about 258 to about 275, wherein at least two consecutive amino acids within the regions of amino acids 129-137, or amino acids 182-189, or amino acids 257-264 as depicted at SEQ ID NO:1 are replaced by an amino acid sequence including Xaaₙ, wherein n is from about 5 to about 21, and each Xaa is independently a random amino acid. The cell is exposed to a pathogen or a toxin, and whether the polypeptide prevents cell death is determined by incubating the cell under conditions such that the pathogen or the toxin kills a cell that does not include a polypeptide that prevents cell death after exposure to a pathogen or a toxin. The presence of a cell that proliferates indicates the polypeptide binds the pathogen, the toxin, a polypeptide, or a polynucleotide. The pathogen may be, for instance, a virus or a microbe. Examples of microbes include a bacterium, a rickettsia, and a fungus. Examples of toxins include a biological toxin or a chemical toxin.

### BRIEF DESCRIPTION OF THE FIGURES

FIG. 1. Amino acid sequence (SEQ ID NO:1) of amino acids 119-275 of the Venezuelan equine encephalitis (VEE) virus capsid polypeptide carboxy terminal portion and a nucleotide sequence (SEQ ID NO:12) that encodes SEQ ID NO:1.

FIG. 2. 2A, Specific examples of members (Adaptein-1 and Adaptein-2) of a collection of polypeptides of the present invention. 2B, an alignment of the Adaptein nucleotide sequences with the CCD nucleotide sequence. A-1, Adaptein-1; A-2, Adaptein-2; CCD, amino acids 119-275 of VEE virus capsid polypeptide carboxy terminal portion; HindIII and XhoI, restriction endonuclease sites; dashes indicate an absence of a nucleotide. FIG. 2C, an alignment of the Adaptein amino acid sequences with the CCD amino acid sequence. Dashes indicate an absence of an amino acid.

FIG. 3. Nucleotide sequence (SEQ ID NO:6) encoding the tat-CCD fusion polypeptide and predicted amino acid sequence (SEQ ID NO:7).

### DETAILED DESCRIPTION OF PREFERRED EMBODIMENTS OF THE INVENTION

Compounds

The present invention provides collections of polypeptides. A collection of polypeptides is also referred to herein as a library, and as an adaptein library. As used herein, “polypeptide” refers to a polymer of amino acids linked by peptide bonds and does not refer to a specific length of a polymer of amino acids. Thus, for example, the terms peptide, oligopeptide, protein, and enzyme are included within the definition of polypeptide. This term also includes post-expression modifications of the polypeptide, for example, glycosylations, acetylations, phosphorylations and the like. In some aspects of the invention, a polypeptide is isolated. As used herein, an “isolated” polypeptide or polynucleotide means a polypeptide or polynucleotide that has been either removed from its natural environment, produced using recombinant techniques, or chemically or enzymatically synthesized. In some aspects of the invention, the polypeptide is preferably purified. As used herein, a “purified” polypeptide or polynucleotide means a polypeptide or polynucleotide that is essentially free from any other polypeptide or polynucleotide and associated cellular products or other impurities. As used herein, a “collection” of polypeptides or polynucleotides is a population of at least two polypeptides or polynucleotides, where the population includes regions of amino acids or nucleotides that are identical in each member of the population, and a region of amino acids or nucleotides that are not identical in each member of the population. Unless otherwise specified, “a,” “an,” “the,” and “at least one” are used interchangeably and mean one or more than one.

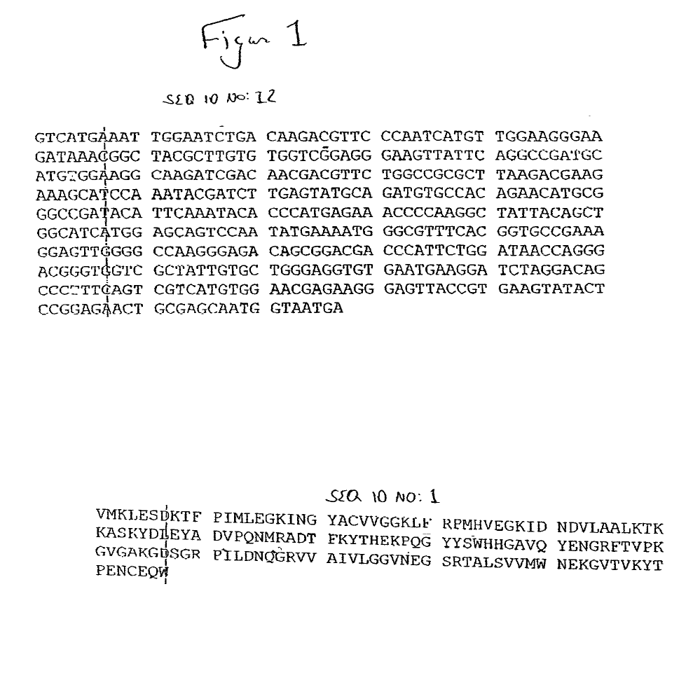

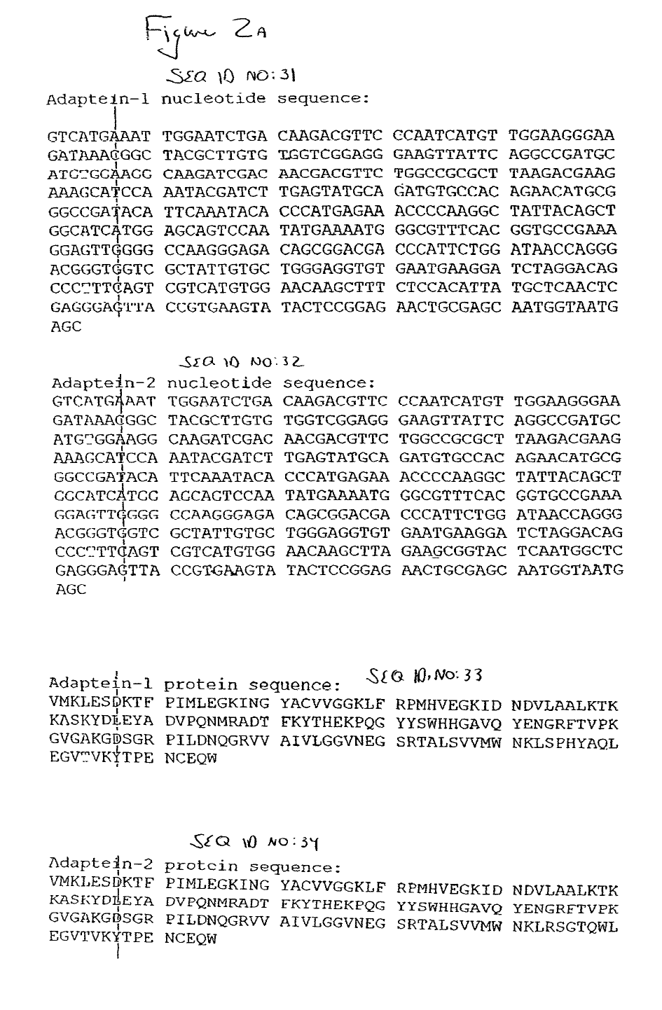

Each polypeptide of a collection includes a fragment of an amino acid sequence having a peptide backbone conformation that acts to display a variable amino acid sequence on the surface of the polypeptide. Variable amino acid sequences are described in greater detail herein. A fragment of an amino acid sequence having this peptide backbone conformation is also referred to herein as a carrier polypeptide or scaffold polypeptide. A preferred example of such an amino acid sequence is a carboxy terminal portion of the Venezuelan equine encephalitis (VEE) virus capsid polypeptide. An example of the amino acid sequence of the VEE capsid polypeptide is available at GenBank Accession Number L01443. A preferred carboxy terminal portion of the VEE capsid polypeptide, also referred to herein as “CCD,” begins at about amino acid 119 and ends at about amino acid 275, and is depicted at SEQ ID NO:1 (see FIG. 1). Amino acids 119-275 of the CCD are encoded by nucleotides 7916-8386 of GenBank Accession Number L01443, and are shown in FIG. 1. This amino acid sequence forms a trypsin resistant and chymotrypsin-resistant structure of predominantly P-sheets, with small loops connecting sequential strands.

Accordingly, in one aspect of the invention, a collection of polypeptides includes at least two polypeptides, where each polypeptide includes a fragment of SEQ ID NO:1. The fragment begins at any amino acid from about 119 to about 124, preferably, about 119. The fragment ends at any amino acid from about 258 to about 275, preferably, about 275. The fragment further includes a variable amino acid sequence, which is described in detail herein. The variable amino acid sequence replaces from about 1 to about 4, more preferably, from about 2 to about 3, most preferably, about 2, amino acids within 1 of 3 regions of the fragment. The 3 regions are amino acids 129-137, amino acids 182-189, and amino acids 257-264 of SEQ ID NO:1. Preferably, a variable amino acid sequence replaces amino acids within the third region, i.e., amino acids 257-264. Preferably, the amino acids within the third region that are replaced by the variable amino acid sequence are amino acids 260-261 of SEQ ID NO:1.

Another example of a fragment of an amino acid sequence having a peptide backbone conformation that acts to display a variable amino acid sequence on the surface of the polypeptide is the inactive staphylococcal nuclease (SNase) polypeptide (KETAAAKFERQHMDSSTSAASSSNYCNQMMKSRNLTKDRCKPVNTFVHES LADVQAVCSQKNVACKNGQTNCYQSYSTMSITDCRETGSSKYPNCAYKTT QANKHIIVACEGNPYVPVHFAASV, SEQ ID NO:8, depicted at GenBank Accession Number 3402176). The variable amino acid sequence replaces from about 1 to about 4 amino acids from the region of amino acids 19-27 of SEQ ID NO:8.

The variable amino acid sequence that is used to replace amino acids of a fragment of SEQ ID NO:1 has the amino acid sequence Xaaₙ. The “n” can be, in increasing order of preference, from about 5 to about 21, from about 6 to about 18, from about 6 to about 12, most preferably, about 6. Each Xaa is independently any amino acid, preferably one of the 20 natural amino acids. Thus, in a single collection of polypeptides, each member of the collection has a variable amino acid sequence that has the same number of amino acids (i.e., about 5 to about 21) but a different amino acid sequence. Accordingly, in the aspect of the present invention where amino acids 260-261 are replaced by Xaa, and n is 6, a polypeptide of a collection of polypeptides has the following amino acid sequence: amino acids 119-259 as depicted at SEQ ID NO:1 followed by the amino acid sequence XaaXaaXaaXaaXaaXaa (SEQ ID NO:11) followed by amino acids 262-275 as depicted at SEQ ID NO:1.

The present invention is also directed to individual members of the collections of polypeptides. The number of members of a collection of polypeptides is large and varies as a function of the value of “n” in the variable amino acid sequence Xaaₙ. The number of members of a collection of polypeptides is also finite, and the amino acid sequence of each member can be readily determined by one skilled in the art by methodically changing one amino acid at a time in the variable amino acid sequence.

Typically, the variable amino acid sequence is flanked on either side by two amino acids. These two amino acids on each side are present as a result of introducing 2 restriction endonuclease sites into the nucleotide sequence encoding a polypeptide of the present invention. For instance, when the restriction endonuclease is HindIII, the 2 amino acids will be KL, and when the restriction endonuclease is XhoI, the 2 amino acids will be LE.

Optionally and preferably, the variable amino acid sequence is flanked by a linker amino acid sequence. A linker can be used to provide some conformational freedom to the variable amino acid sequence, and/or to allow the variable amino acid sequence to be spatially separated from the fragment amino acid sequence. Preferably, a linker includes from about 1 to about 4 amino acids, more preferably, about 2 to about 3 amino acids, most preferably, 3 amino acids. Examples of linkers include Ser-Ser-Gly, Ser-Gly-Ser, Gly-Ser-Gly, and Ser-Gly.

Optionally and preferably, each polypeptide of a collection of polypeptides also includes a cell-permeant region. As used herein, a “cell-permeant region” is a polypeptide that causes polypeptides to which it is fused to traverse cell membranes, including cultured cells, and cells present in animals including cells of the blood-brain barrier (see, for instance, Nagahara et al., Nat. Medicine, 4, 1449-1452 (1998), Schwarze et al., Science 285, 1569-1572 (1999), and Vocero-Akbani et al., Meth. Enzym., 322, 508-521 (2000)). As used herein, two polypeptides are “fused” when they are covalently bound by a peptide bond. Preferably, the cell-permeant region is present at the amino terminal end of the polypeptide, fused to the amino terminal amino acid of a fragment amino acid sequence. Preferred examples of cell-permeant regions include YGRKKRRQRRR (SEQ ID NO:2), RQIKIWFQNRRMKWKK (SEQ ID NO:3), RQIKIWFPNRRMKWKK (SEQ ID NO:4), and RQPKIWFPNRRPKWKK (SEQ ID NO:5), preferably, the cell-permeant region is SEQ ID NO:2.

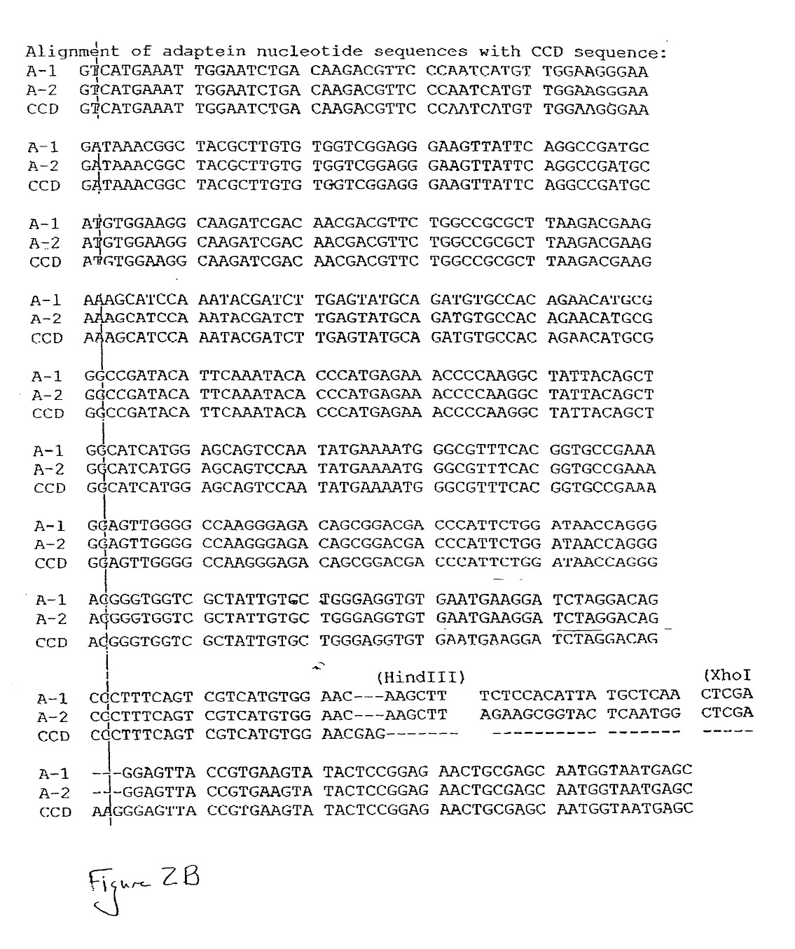

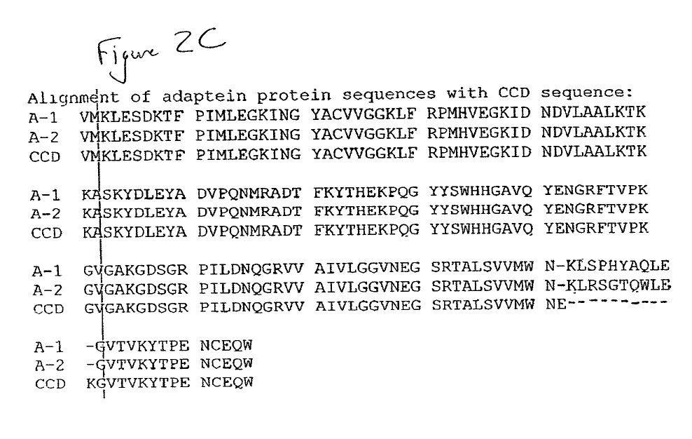

Specific examples of members of one collection of polypeptides include the amino acid sequences depicted in FIG. 2. The two examples, labeled Adaptein-1 and Adaptein-2, each contain a variable amino acid sequence of 6 amino acids. In Adaptein-1, the sequence of the variable amino acid sequence is SPHYAQ (amino acids 262-267 of SEQ ID NO:33), and in Adaptein-2, the sequence of the variable amino acid sequence is RSGTQW (amino acids 262-267 of SEQ ID NO:34). The amino acids KL and LE which flank the variable amino acid sequences are encoded by the nucleotides encoding the restriction endonuclease sites HindIII and XhoI, respectively.

The present invention also includes a population of cultured cells including two or more cells, where each cell of the population includes one polypeptide of one collection of polypeptides. The present invention also provides individual cultured cells containing a polypeptide that is a member of a collection of polypeptides. The cells may be prokaryotic or eukaryotic, preferably, eukaryotic, more preferably, vertebrate, most preferably, mammalian. Examples of useful mammalian cultured cells include 293 cells, macrophage cells, including J774 and RAW 264.7, HeLa cells, and Vero cells, each of which is available from the ATCC

The present invention also provides collections of polynucleotides. As used herein, “polynucleotide” refers to a polymeric form of nucleotides of any length, either ribonucleotides or deoxynucleotides, and includes both double- and single-stranded DNA and RNA. A polynucleotide may include nucleotide sequences having different functions, including, for instance, coding sequences, and non-coding sequences such as regulatory sequences. A polynucleotide can be obtained directly from a natural source, or can be prepared with the aid of recombinant, enzymatic, or chemical techniques. A polynucleotide can be linear or circular in topology, and can be, for example, a portion of a vector, such as an expression or cloning vector, or a fragment.

A collection of polynucleotides encodes a collection of polypeptides of the present invention. Thus, each member of a collection of polynucleotides includes a coding sequence that encodes a fragment of an amino acid sequence, preferably SEQ ID NO:1. A “coding sequence” is a nucleotide sequence that encodes a polypeptide and, when placed under the control of appropriate regulatory sequences, expresses the encoded polypeptide. The boundaries of a coding region are generally determined by a translation start codon at its 5′ end and a translation stop codon at its 3′ end.

The coding sequence further includes a nucleotide sequence encoding a variable amino acid sequence. Each codon of the coding sequence encoding the variable amino acid sequence may have the nucleotide sequence (NNN)ₘ. The “m” can be, in increasing order of preference, from about 5 to about 21, from about 6 to about 18, from about 6 to about 12, most preferably, about 6. Each “N” is independently either adenine (A), thymine (T), guanine (G), or cytosine (C). Preferably, each codon of the coding sequence encoding the variable amino acid sequence has the nucleotide sequence (NNZ)ₘ. “Z” is independently either R, Y, M, K, or S, where R is G or A, Y is T or C, M is A or C, K is G or T, and S is G or C. Most preferably, each codon of the coding sequence encoding the variable amino acid sequence has the nucleotide sequence (NNK)ₘ.

The present invention is also directed to individual members of the collections of polynucleotides. The number of members of a collection of polynucleotides is large and varies partially as a function of the value of “m” in (NNN)ₘ. The number of members of a collection of polynucleotides is also finite, and the amino acid sequence of each member can be readily determined by one skilled in the art. Specific examples of members of such a collection include the nucleotide sequences depicted in FIG. 2.

Optionally and preferably, a coding sequence of a polynucleotide of the present invention further includes a nucleotide sequence encoding a linker as described herein. Optionally and preferably, a coding sequence of a polynucleotide of the present invention also includes a nucleotide sequence encoding a cell-permeant region as described herein.

The present invention also includes a population of cultured cells including two or more cells, where each cell of the population includes one polynucleotide of one collection of polynucleotides. The present invention also provides individual cultured cells containing a polynucleotide that is a member of a collection of polynucleotides. Preferably, when a polynucleotide of the present invention is present in a eukaryotic cell, the polynucleotide is inserted into the genomic DNA of the cell.

A polynucleotide of the invention can be inserted in a vector. A vector can be used in the construction of the collection of polynucleotides described herein, as a way to insert polynucleotides of the present invention into the genomic DNA of a eukaryotic cell, and/or as a way to cause a polynucleotide of the present invention to express a polypeptide of the present invention.

A vector is a replicating polynucleotide, such as a plasmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual., Cold Spring Harbor Laboratory Press (1989) or Ausubel, R. M., ed. Current Protocols in Molecular Biology (1994). A vector can provide for further cloning (amplification of the polynucleotide), i.e., a cloning vector, or for expression of the polypeptide encoded by the coding region, i.e., an expression vector. A vector can provide for the insertion of a polynucleotide into the genomic DNA of a eukaryotic cell, i.e., a suicide vector. A vector can provide for more than one of these functions, for instance, a vector can provide for expression and be a suicide vector. The term vector includes, but is not limited to, plasmid vectors, viral vectors (including retroviral vectors), cosmid vectors, or artificial chromosome vectors. Typically, a vector is capable of replication in a bacterial host, for instance E. coli.

Selection of a vector depends upon a variety of desired characteristics in the resulting construct, such as a selection marker, vector replication rate, and the like. Suitable host cells for cloning or expressing the vectors herein are prokaryote or eukaryotic cells. Preferably the host cell secretes minimal amounts of proteolytic enzymes. Suitable prokaryotes include eubacteria, such as gram-negative or gram-positive organisms, for example, E. coli, Bacilli such as B. subtilis, Pseudomonas species such as P. aeruginosa, or Salmonella typhimurium. Preferably, E. coli is used.

An expression vector optionally includes regulatory sequences operably linked to the coding region. A “regulatory sequence” is a nucleotide sequence that regulates expression of a coding sequence to which it is operably linked. Nonlimiting examples of regulatory sequences include promoters, enhancers, transcription initiation sites, translation start sites, translation stop sites, transcription terminators, and poly(A) signals. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

The invention is not limited by the use of any particular promoter, and a wide variety are known. Promoters act as regulatory signals that bind RNA polymerase in a cell to initiate transcription of a downstream (3′ direction) coding region. The promoter used in the invention can be a constitutive or an inducible promoter. It can be, but need not be, heterologous with respect to the host cell. Promoter sequences are known for eukaryotes and can be suitably inserted into an expression vector. Transcription of a coding sequence encoding a polypeptide of the present invention in a host cell can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, and Hepatitis-B virus. Preferably, the promoter is the human cytomegalovirus (CMV) immediate early promoter.

Transcription of a coding sequence encoding a polypeptide of the present invention by a eukaryote cell can be increased by inserting an enhancer sequence into the vector. Enhancers are cis-acting elements of DNA, usually having about 10 to 300 nucleotides, that act on a promoter to increase its transcription. Enhancers are relatively orientation- and position-independent, having been found 5′ and 3′ to coding regions, within an intron as well as within the coding sequence itself. Many enhancer sequences are now known from mammalian genes (globin, elastase, albumin, alpha-fetoprotein, and insulin). Enhancers from eukaryotic cell viruses are also known and include the SV40 enhancer on the late side of the replication origin, the cytomegalovirus early promoter enhancer, the polyoma enhancer on the late side of the replication origin, adenovirus enhancers, and the enhancer present in the Moloney murine sarcoma virus 5′ long terminal repeat. The enhancer may be spliced into the vector at a position 5′ or 3′ to the coding region encoding a polypeptide of the present invention, but is preferably located at a site 5′ of the promoter.

The polynucleotide used to transform the host cell optionally includes one or more marker sequences, which typically encode a molecule that inactivates or otherwise detects or is detected by a compound in the growth medium. For example, the inclusion of a marker sequence can render the transformed cell resistant to an antibiotic, or it can confer compound-specific metabolism on the transformed cell. Examples of a marker sequence are sequences that confer resistance to kanamycin, ampicillin, chloramphenicol, tetracycline, puromycin, neomycin, and formulations of phleomycin D1 including, for example, the formulation available under the trade-name ZEOCIN (Invitrogen).

The present invention also includes crystals of a polypeptide having the amino acid sequence depicted at SEQ ID NO:1, which are suitable for x-ray crystallographic analysis. Preferably, the crystals have a P2₁2₁2₁ space group. Most preferably, the crystal includes rectangular shaped unit cells, each unit cell having the dimensions a=about 69.3 to about 72.0 Å, preferably about 70.0 Å, b=about 75.2 to about 76.0 Å, preferably about 75.0 Å, and c=about 90.1 to about 95.0 Å, preferably about 94.7 Å, α=about 90, β=about 90, and γ=about 90. The crystallized enzyme is a monomer and has three molecules in the asymmetric unit. Variation in buffer and buffer pH as well as other additives such as polyethylene glycol (PEG) is apparent to those skilled in the art and may result in similar crystals.

The present invention is also directed to methods for crystalizing a polypeptide. The polypeptide may have the amino acid sequence depicted at SEQ ID NO:1, or be a polypeptide of the present invention.

A method for crystalizing a polypeptide includes growing crystals in hanging drops at about 18° C. using about 3 mg/ml to about 20 mg/ml of polypeptide, preferably, about 10 mg/ml mixed with about an equal volume of precipitant. The volume of polypeptide used is about 1 μl to about 15 μl, preferably, about 5 μl. Two different crystallization conditions can be used to produce crystals. In one condition, about 20% to about 28%, preferably 24% (w/v) polyethylene glycol (PEG), about 0.05 M to about 0.2 M, preferably, about 0.1 M ammonium sulfate, about 1 mM to about 20 mM, preferably, about 5 mM urea, and about 0.02 M to about 0.3 M, preferably, about 0.1 M Bis-Tris buffer, pH about 6 to about 8, preferably, about pH 6.5 can be used. Preferably, the PEG is PEG 1000. In the second condition, about 15% to about 25%, preferably, about 20% (w/v) PEG 4000, about 0.05 M to about 0.4M, preferably, about 0.2 M MgCl₂ buffered in Bis-Tris, pH about 6 to about 8, preferably, about pH 6.5 can be used. The crystals in each condition can be allowed to grow for about 8 to about 12 days, preferably, about 10 days. Crystals can be transferred to precipitant solution containing about 5% to about 25%, preferably, about 10% 2,4-methylpentanediol (MPD) as cryo-protectant.

Methods of Use

The present invention is further directed to methods for identifying a polypeptide within a collection. In one aspect, the method identifies a polypeptide within a collection, where the polypeptide prevents cell death after the cell is exposed to a pathogen or a toxin. In another aspect, the method identifies a polypeptide within a cell, where the polypeptide binds a pathogen, a toxin, a polypeptide, of a polynucleotide. The methods include providing a cell that contains a polypeptide of the present invention, exposing the cell to a pathogen or a toxin, and determining if the polypeptide of the present invention prevents cell death. To determine if the polypeptide prevents cell death, the cell is incubated under conditions that result in the pathogen or the toxin killing those cells that do not contain a polypeptide of the present invention, or contain a polypeptide of the present invention that does not protect the cell. The presence of a living cell indicates the polypeptide prevents cell death after exposure to a pathogen or a toxin. Without intending to be limited by theory, it is expected that a variable amino acid sequence of the polypeptides of the present invention will protect a cell from the pathogen or toxin by binding to target polypeptides or nucleotides of either viral or host cell origin. For instance, an amino acid sequence may bind to a pathogen polypeptide or nucleic acid sequence and prevent replication. A variable amino acid sequence may interact with host cell polypeptides or nucleic acids to protect the cell from pathogen challenge. For example, the variable amino acid sequence may selectively inhibit a cellular protease required for viral protein processing, or may down-regulate the expression and/or transport of host cell receptors required for pathogen or toxin entry.

The pathogen or toxin is preferably cytotoxic to the cell. As used herein, “cytotoxic” and “cytopathic” are used interchangeably and refer to the ability of a pathogen or a toxin to cause cell death. Preferably, a pathogen or a toxin are cytolytic, i.e., cause the cell contacted with the pathogen or toxin to lyse. As used herein, “cell death” refers to the permanent cessation of proliferation, and is typically apparent as the degeneration or necrosis of the cell. As used herein, “proliferation” includes, but is not limited to, replication. When the pathogen or toxin is cytotoxic but not cytolytic, cell viability can be determined by methods known to the art, including trypan blue exclusion, propidium iodide exclusion, and observing the presence of colonies growing on a plate containing other cells that have ceased to proliferate. When the pathogen or toxin is cytolytic, cell viability can be evaluated by measuring the amount of cell lysis, and by observing the presence of colonies growing on a plate after exposure to a pathogen or a toxin.

Examples of pathogens include viruses and microbes that are cytotoxic to cells. Without intending to be limiting, examples of viruses include members of the genera within the family Bunyaviridae, such as Bunyavirus, Nairovirus, Phlebovirus, and Hantavirus. Examples of Bunyavirus include LaCrosse virus, Bunyamwera virus, and Oropuche virus. Examples of Nairovirus include Crimean Congo haemorrhagic fever virus and Dugbe virus. Examples of Phlebovirus include Rift Valley fever virus, Punta Toro virus, Sandfly Sicilian virus, and Unkuniemi virus. Examples of Hantavirus include Hantaan virus, Sin Nombre virus, and Seoul virus.

Other non-limiting examples of viruses include members of the flavivirus family, including the genus Hepacivirus (for instance, Hepatitis C virus) and the genus Flavivirus (Yellow fever virus, Tick-borne encephalitis complex viruses), members of the family Togaviridae, genus Alphavirus (eastern equine encephalitis virus, Venezuelan Equine Encephalitis virus, western equine encephalitis virus), members of the family Filovirus (Ebola and Marburg viruses), Equine Morbillivirus (Hendra virus), members of the family Arenavirus (Lassa fever, Junin, Machupo, Sabia, Flexal, and Guanarito viruses), Variola major virus (Smallpox virus), human immunodeficiency virus, foot and mouth disease virus, and influenza virus.

Examples of microbes that can be used in the methods described herein include, for instance, bacteria and fungi. Non-limiting examples of bacteria include rickettsias, including members of the genera Rickettsias (e.g., Rickettsia prowazekii, and Rickettsia rickettsii) and Coxiella (e.g., Coxiella burnetii). Other examples of bacteria include members of the Enterobacteriaceae (including Escherichia coli, Shigella spp., Salmonella spp., and Yersinia spp.), Brucella spp. (for instance, Brucella abortus, Brucella melitensis, and Brucella suis), Bacillus anthracis, Burkholderia spp. (including Burkholderia mallei, and Burkholderia pseudomallei, Clostridium botulinum, Francisella tularensis. Non-limiting examples of fungi include, for instance, Coccidioides immitis.

In many cases, strains of pathogens that are used as vaccines can also be used. Examples include the Bacillus anthracis Sterne strain (Brossier et al., Infect. Immun., 68, 1781-1786 (2000)), the MP-12 live-attenuated vaccine strain of Rift Valley fever virus (Caplen et al., J. Gen. Virol., 66, 2271-2277 (1985)), Yellow fever virus vaccine strain 17D, the TC-83 attenuated vaccine strain of Venezuelan equine encephalitis virus.

Examples of toxins include biological toxins and chemical toxins. As used herein, a “biological toxin” is a toxin that is produced by a cell. Non-limiting examples of biological toxins include, for instance, Abrin, Aflatoxins, Anthrax toxin, Botulinum toxins, Clostridium perfringens epsilon toxin, Conotoxins, Diacetoxyscirpenol, Ricin, Saxitoxin, enterotoxins, Shigatoxin, Staphylococcal enterotoxins, Tetrodotoxin, and T-2 toxin. Examples of chemical toxins include, for instance, sarin.

The cell that is exposed to a pathogen or toxin can be ex vivo or in vivo, preferably, ex vivo. As used herein, “in vivo” refers to a cell that is present within the body of an animal. As used herein, “ex vivo” refers to a cell that has been removed from the body of an animal. Ex vivo cells include, for instance, primary cells (e.g., cells that have recently been removed from a subject and are capable of limited growth in tissue culture medium), and cultured cells (e.g., cells that are capable of long term culture in tissue culture medium). The cell is a eukaryotic cell, preferably, a vertebrate cell, most preferably, a mammalian cell. Examples of mammalian cells include human, as well as other animals (for instance, mice, rats, or hamsters) that can be used as animal models in the study of the protective ability of polypeptides of the present invention.

Cells that are useful in the methods described herein vary depending on the pathogen or toxin used. The cell chosen for use with a particular pathogen or a particular toxin is one for which the pathogen or toxin is cytotoxic, preferably, cytolytic. Which cells are appropriate for use with a particular pathogen or a particular toxin is known to the art, and can be chosen by a person having skill in the art. Examples of some pathogen/cell combinations include Bacillus anthracis and the macrophage cell line J774A.1 (Friedlander et al., Infect. Immun., 61, 245-252 (1993), and Hanna et al., Mol. Biol., Cell, 3, 1269-1277 (1992)), Rift Valley fever virus and Vero cells, Venezuelan equine encephalitis and Vero cells, and Rickettsia spp. and primary chick embryo cells (Walker and Cain, Lab Invest., 43, 388-396 (1980)) and human endothelial cell culture (Walker et al., Fed. Proc., 40, 72A (1981)).

In one aspect of the invention, the polypeptide of the present invention can be expressed in a cell that is to be exposed to a pathogen or a toxin. Typically, the polypeptide will be present in a cell in a vector as described herein, preferably inserted into the chromosome. In another aspect, the polypeptide of the present invention can be introduced to the cell. Typically, such a polypeptide will contain a cell-permeant region that will allow the polypeptide to traverse the cell membrane. In this aspect, the polypeptide can be introduced to the cell before, at the same time, or after exposing the cell to the pathogen or toxin.

After protective polypeptides are identified by these methods, the polynucleotides encoding the protective polypeptides can be cloned and expressed. The expressed protective polypeptides may be isolated using chromatographic methods known to the art, preferably using cation exchange and size exclusion chromatography. Isolated protective polypeptides can then be introduced to cells to verify that the polypeptides are protective. Optionally, the ability of the isolated protective polypeptides can be introduced to animals to evaluate whether the protective polypeptides will protect against challenge with a pathogen.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Crystalization of the CCD Protein

Cloning, expression and purification of CCD. cDNAs encoding the capsid C-terminal domain (CCD; capsid residues 118-275) of the VEE virus were amplified from purified pUC18 plasmid containing the Trinidad donkey strain of VEE virus (Kinney et al., Virology, 170, 19-30 (1989)) using PCR and the primers 5′-GGGAATTCCATATGGTCATGAATTGGAATCTGACAAG (SEQ ID NO:9) and 5′-GAATTCGGATCCTCATTACCATTGCTCGCAGTTCTCCGGAGT (SEQ ID NO:10). The PCR products were each digested with Nde1 and BamHI restriction enzymes (Promega, Madison, Wis.) and subcloned into pET30 expression vectors (Novagen, Madison, Wis.).

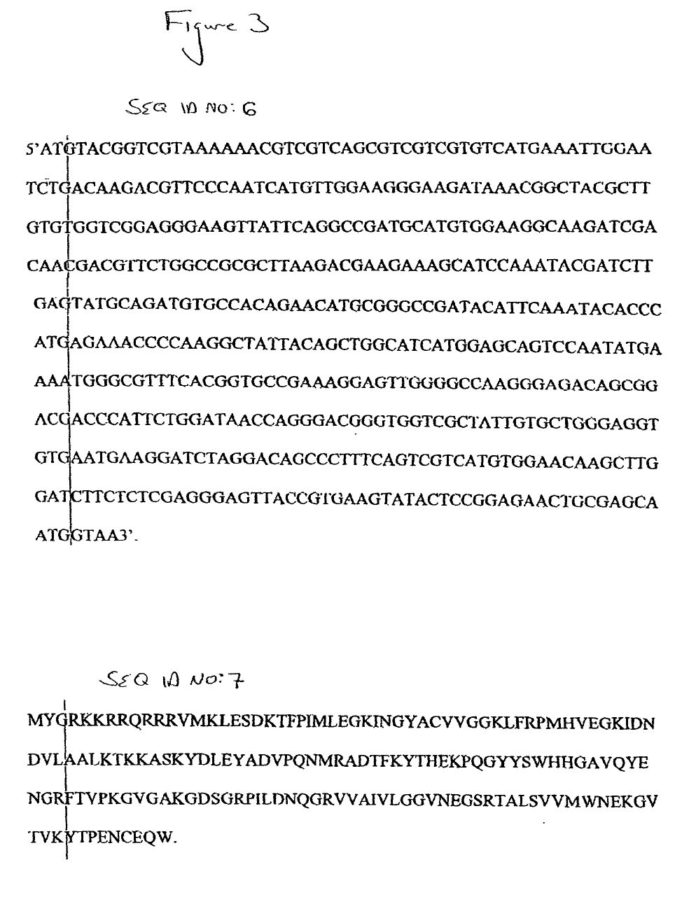

The tat-CCD construct was produced by PCR using CCD in the pET30 vector and the primers N-TATCCD(5′ATGTACGGTCGTAAAAAACGTCGTCAGCGTCGTCGTGTCATGAAATTG GAATCTGACA3′) and CBAM-VEE(5′GAATTCGGATCCTCATTACCATTGCTCGCAGTTCTCCGGAGT3′). The PCR product was phenol-chloroform extracted and was ligated into the pETBLUE vector. It was then transformed into NovaBlue Singles (Novagen) and plated on LB-Bluogal-IPTG-carbenicillin-tetracycline plates. White colonies were selected for amplification, plasmid purification, and sequencing. The tat-CCD cDNA sequence was determined and is depicted in FIG. 3.

The VEE virus CCD was expressed from BL21(DE3) cells grown at 37° C., induced with 1 mM isopropyl-thio-β-D-galactoside (IPTG) for 2 to 4 hours and recovered from the supernatant after mild sonication of bacterial pellets in ice-cold lysis buffer (20 mM TrisHCl pH 7.3, 5 mM DTT, 150 mM NaCl, 5% glycerol, 2 mM EDTA). The CCD was purified using cation exchange HPLC (Poros 20 SP column, Perseptive Biosystems, Farmingham, Ma.) in chilled MOPS buffer (20 mM MOPS pH 7.3, 5 mM DTT) and size exclusion chromatography (Biosep SEC-S3000, Phenomenex, Torrence, Calif.) in chilled Tris buffer (10 mM Tris pH 7.3, 150 mM NaCl, 10 mM DTT). Purified CCD was concentrated to 30 mg/ml using ultrafiltration (Millipore, Bedford, Ma.).

Crystallization and data collection. Crystals of the CCD were grown in hanging drops at 18° C. using 5 μl of protein solution (10 mg/ml) mixed with an equal volume of precipitant. Two different crystallization conditions produced crystals that diffract to high resolution. Condition one, producing crystal type I, was 24% (w/v) polyethylene glycol (PEG) 1000, 0.1 M ammonium sulfate, 5 mM urea and 0.1 M Bis-Tris buffer pH 6.5. Condition two, producing crystal form II, was 20% (w/v) PEG 4000, 0.2 M MgCl₂ buffered in Bis-Tris at pH 6.5. PEG 4000 could not be replaced with PEG 1000 in condition two. CCD crystals in both conditions grew to approximately 0.2 mm×0.2 mm×0.5 mm size within 10 days.

Crystals were transferred to precipitant solution containing 10% 2-methyl dioxypentane (MPD) as cryo-protectant. X-ray diffraction data were collected at 105 K using MacScience Dip 2030 imaging plate detector on a rotating anode generator running at 50 kV and 90 mA. All the data were processed using the DENZO and SCALEPACK programs (Otwinowski & Minor, Methods Enzymol., 276, 307-326 (1997)). Details of the data collection and quality of the data were given in Table 1.

TABLE 1
Data collection statistics for VEE virus CCD crystals.

| | Crystal form I | Crystal form II |
|---|---|---|
| Spacegroup | P2₁2₁2₁ | P2₁2₁2₁ |
| Unit cell (a, b, c) (Å) | 69.3, 75.2, 94.7 | 72.0, 76.0, 90.1 |
| Temperature (K) | 105 | 105 |
| Crystal to detector (mm) | 180 | 200 |
| Oscillation angle (degree) | 1 | 1 |
| Resolution range (Å) | 30-2.3 | 30-2.45 |
| No. of observations | 168,290 | 172,603 |
| No. of unique observations | 22,453 | 18,598 |
| Overall completeness (%) | 99.0 | 99.2 |
| Overall R_{merge}^{#} (%) | 6.9 | 7.0 |
| Final shell (Å) | 2.38-2.3 | 2.54-2.45 |
| Final shell completeness (%) | 97.9 | 99.8 |
| Final shell R_{merge}^{#} (%) | 30.9 | 26.2 |

Molecular replacement solution. The calculated Matthew coefficient implied, and subsequent molecular replacement solutions (Navaza, J. Acta Crystallog., A50, 157-163 (1994)) confirmed, that there are three monomers (A, B, and C) per asymmetric unit. The CCD structure in crystal type I was solved by molecular replacement with the AMoRe program (Navaza, J. Acta Crystallog., A50, 157-163 (1994)) contained in the CCP4 suite. The C-terminal domain from Sindbis virus capsid protein (PDB code 1WYK; Choi et al., Nature, 354, 37-43 (1991)) was used as a search model. Two prominent peaks were obtained in the cross-rotation search calculated using reflection data from 10 Å to 3 Å and with an integration radius of 30 Å. The largest peak in the cross-rotation function gave rise to the largest peak in the translation function using P2₁2₁2₁ as the space group. The position of this monomer was fixed and the translation of the second monomer determined. This translation was found with the second highest peak in the rotation function. With the first and second monomers fixed, the translation function for the third monomer was obtained. This translation vector corresponded to the 39th rotation function peak. After rigid body refinement performed in AMoRe, the crystallographic R-value was 44% with a correlation coefficient of 0.50 calculated with data between 8 Å and 3 Å resolution.

Structure refinement. Refinement of the CCD structure in crystal type I was performed using the Crystallography and NMR System package (CNS version 0.5; Brunger et al., Acta Criystallogr, D54, 905-921 (1998)), with maximum likelihood target function. Bulk solvent correction and overall anisotropic temperature factors were applied during refinement. The initial model was subjected to torsion angle dynamics simulated annealing with a starting temperature of 4,000K and a cooling step interval of 25K. Following simulated annealing, repeated rounds of Powell positional refinement followed by individual B-factor refinement and model building with the graphic package O (Jones et al., Acta Crystallog., A47, 110-119 (1991) were performed. The VEE capsid sequence was incorporated into the model using sigma-A-weighted Fo-Fc electron density maps (Read, Acta Crystallog., A42, 140-149(1986)). The first six amino-terminal residues in monomers A and B were also built into sigma-A-weighted Fo-Fc electron density maps. Included in the crystal type I structure are seven urea molecules and two sulfate ions. The possibility that these are water molecules was eliminated by examination of difference Fourier maps. These difference maps, calculated with water in place of urea or sulfate, had significant positive density on and around the replaced water molecules. Details of the refined model are given in Table 2.

Refinement of the CCD structure in crystal type II was initiated with CNS rigid body refinement. The starting model was the refined crystal type I structure. Following rigid body minimization, the type II model was refined as described above. No non-crystallographic symmetry restraints were applied during structure refinement. For both structures, 5% of the diffraction data were excluded from the refinement calculations and used to calculate the free R-value (Brunger, Nature, 355, 472-474(1992)). The final R-factors for both structures are given in Table 2.

TABLE 2
Refinement statistics for VEE virus CCD structures.

| | Crystal form I | Crystal form II |
|---|---|---|
| Resolution range (Å) | 30-2.3 | 30-2.45 |
| R_{cryst} (R_{free}) (%) | 22.8 (26.3) | 21.6 (25.6) |
| No. of reflections (test | 20,772 (1069) | 17,464 (896) |
| set) | | |
| Residues | Molecule A: 120-275 | Molecule A: 119-275 |
| | Molecule B: 119-275 | Molecule B: 119-275 |
| | Molecule C: 124-275 | Molecule C: 123-275 |
| No. of water molecules | 176 | 223 |
| No. of urea molecules | 7 | — |
| No. of sulfate molecules | 2 | — |
| Average B-factors (Å²) | | |
| CCD monomers | A: 26; B: 29; C: 44 | A: 27; B: 28; C: 37 |
| solvent | 36 | 34 |
| urea | 50 | — |
| sulfate | 76 | — |
| Bond length rmsd¹ (Å) | 0.007 | 0.007 |
| Bond angle rmsd(°) | 1.35 | 1.38 |

¹rmsd, root mean square deviation.

### Example 2

### Cloning Combinatorial Adaptein Libraries into Packaging Vectors

This describes the insertion of a DNA oligonucleotide, which contains a stretch of random sequence, into the DNA sequence coding for the tat-CCD protein, within a retrovirus packaging vector. This allows for the expression of a fusion protein that contains tat, CCD, and a random peptide inserted into the CCD sequence. A number of approaches are being used to create these adaptein combinatorial libraries within packaging vectors.

1. Combinatorial synthetic oligonucleotide method for cloning combinatorial adaptein libraries into packaging vectors.

The tat-CCD DNA sequence was inserted into a number of retroviral packaging vectors, including pLPCX and pLNCX2 from Clontech (Palo Alto, Calif.) and pFB from Stratagene (La Jolla, Calif.). To insert the tat-CCD sequence into pLNCX2, the tat-CCD sequence was amplified by PCR from tat-CCD in the pETBlue plasmid (Novagen, Madison, Wis.). The nucleotide sequence encoding CCD was amplified and ligated into pETBlue as follows. The 5′ fragment of the tat-CCD was amplified using the primers CCDnEcoR(+) (5′AGCTAGGAATTCGGATCCCATATGTACGGTCGTAAAAAACGTC (SEQ ID NO:13) and CCDnHind(−) (5′CTAGCTAAGCTTGTTCCACATGACGACTGAAAG (SEQ ID NO:14). The PCR product of the 5′ fragment was digested with EcoRI (New England Biolabs, Beverly, Mass.) and HindIII (New England Biolabs) and was gel purified. The CCDnEcoR(+) primer added the nucleotides encoding the tat protein to the amino terminal end of the CCD. The pLNCX2 vector was digested with HindIII, partially digested with EcoRI, the largest fragment (4.6 kb) was gel purified, ligated to the 5′ tat-CCD PCR fragment and transformed into DH5α cells for plasmid amplification and subsequent purification. The 3′ fragment was amplified using the primers CCDcNot(−) (5′CTAGCTGCGGCCGCTCATTACCATTGCTCGCAGTTC (SEQ ID NO:15)) and CCDcHindXho(+) (5′AGCTAGAAGCTTGGATCTTCTCTCGAGGGAGTTACCGTGAAGTATAC (SEQ ID NO:16)), which also inserted a HindIII/XhoI cloning site into ccd (between amino acids 260-262 of the full length ccd protein). The above plasmid and the tat-CCD 3′ fragment PCR product were digested with HindIII and NotI (New England Biolabs), were gel purified, and ligated together, thereby creating pLNCX2:tat-CCD that contains a HindIII/XhoI cloning site. To insert the tat-CCD into pFb and pLPCX, the pFb or pLPCX and pLNCX2:tat-CCD were digested with EcoRI and NotI followed by gel purification and ligation of tat-CCD into pFb or pLPCX. In addition, the HindIII site in these tat-CCD expression vectors was converted to BAMHI. This was accomplished by PCR of tat-CCD using the primers bamccdr (5′GATCCTCGAGAGAAGATCCGGATCCGTTCCACATGACGACTGAAAGG GCT (SEQ ID NO:17)), which converted the HindIII site to BamHI and ccd5r1 (5′GATCGAATTCCACCAGCAGAATCGACATATGTACGGTCGTAAAAAAC GTCG (SEQ ID NO: 18)), which inserted a murine leukemia virus ribosomal binding site just 5′ to the tat-CCD start site. This PCR product and pFb:tat-CCD were digested with EcoRI and XhoI (New England Biolabs) and were ligated together to from pFb:tat-CCD:Bam. In addition, the tat-CCD was excised from PFb:tat-CCD:Bam using SalI (Boehringer Mannheim, Indianapolis, Ind.) and Not 1 and was inserted into pLPCX:tat-CCD that had been digested with Xho I (New England Biolabs) and Not I, thereby forming pLPCX:tat-CCD:BAM.

The random library sequence is inserted into the multiple cloning site in the ccd sequence of the above constructs as follows. For each library, three oligos are 5′ phosphorylated and PAGE purified (Biosource International, Camarillo, Calif.). These included LIB (5′AGCTTTCCGGTGGT(NNK)mGGTGGTTCCC (SEQ ID NO:19)), Link P3 (5′ ACCACCGGAA (SEQ ID NO:20)), Link P4 (5′TCGAGGGAACCACC (SEQ ID NO:21)), and Link P5 (5′AGCTGGGAACCACC (SEQ ID NO:22)), where m=the number of amino acids to be in the random library, N=A, T, C, G, and K=T, G. To create a construct to insert into the HindIII/XhoI site of the tat-CCD expression vectors, LIB, Link P3 and Link P4 are annealed together. The binding of Link P3 to the 5′ end of LIB created a HindIII cohesive end, while the binding of Link P4 to the 3′ end of LIB created an XhoI cohesive end. Another library construct for insertion into the HindIII site of the expression vector alone is produced by annealing LIB to Link P3 and Link P5. Lastly, since the tat-CCD expression plasmid was also engineered to contain a BamHI site in place of the HindIII, a construct is generated to insert into the BamHI and XhoI site. The annealed oligos for this construct were LIB-BAM (5′GATCCTCCGGTGGT(NNK)mGGTGGTTCCC (SEQ ID NO:23)), Link P6 (5′ACCAACCGGAG (SEQ ID NO:24)), and Link P4 where Link P6 bound to the 5′ end of LIB-BAM and formed a BamHI cohesive end and Link P4 bound to the 3′ end of LIB-BAM and formed an XhoI cohesive end.

Additional approaches to constructing the insert containing the library are also being used. One of these approaches involves annealing a negative strand of LIB (termed LIB r/c) to LIB itself. The LIB r/c sequence was (5′TCGAGGGAACCACC(MNN)mACCACCGGAG (SEQ ID NO:25)), where M=C, A. When LID and LID r/c were annealed, cohesive ends for BamHI and XhoI are formed. Another approach is to use Sequenase V 2.0 (USB, Cleveland, Ohio) to synthesize the negative LIB strand. The oligos for this are LIBSEQBAM (5′GCACGGATCCTCCGGTGGT(NNK)oGGTGGTTCCCTCGAGATCG (SEQ ID NO:26)) and SEQBAM Rev (5′CGATCTCGAGGGAACCATC (SEQ ID NO:27)). This sequenase product is then digested with BamHI (Promega, Madison, Wis.), and XhoI for insertion into the tat-CCD:BAM expression vectors.

The above constructs are inserted into the tat-CCD expression vectors as follows. The tat-CCD or tat-CCD:BAM expression vectors are digested overnight at 37° C. with the appropriate enzymes (HindIII, BamHI, and/or XhoI) according to the manufacturers' protocol. The restriction digest products are electrophoresed on a 1% agarose gel containing 0.1 mg/ml ethidium bromide. DNA is visualized by ultraviolet light and the appropriate band is excised and gel purified using a QIAquick gel extraction kit (Qiagen, Valencia, Calif.). The purified tat-CCD expression vector is then incubated overnight at 16° C. with the appropriate annealed constructs in the presence of 10×DNA ligase buffer (New England Biolabs), 400 U of T4 DNA Ligase (New England Biolabs), and dH₂0 to a final volume of 20 μl. In addition, the ligated constructs that contained single stranded DNA are subjected to sequenase treatment (0.2 U/μl sequenase, 0.2 mM dNTPs (Sigma, Saint Louis, Mo.), and 5×sequenase buffer (USB), for 1 hour at 37° C.). The ligated library construct and tat-CCD expression vector are transformed into ElectroTen-Blue electroporation competent cells (Stratagene) or XL10-Gold ultracompetent cells (Stratagene) according to the manufacturer's protocol. A small aliquot of cells are plated on LB-carbenicillin agar to determine transformation efficiency. The bacteria containing the library are then expanded and the DNA isolated by standard methods.

2. PCR-based method for cloning a combinatorial adaptein libraries into packaging vectors.

Another strategy for constructing the adaptein library was to use the polymerase chain reaction (PCR) in conjunction with a degenerate oligonucleotide primer to introduce the random sequences into the retroviral packaging vector. A partially degenerate primer of sequence 5′-CCAGGCAAGCTTTCTGGANNKNNKNNKNNKNNKNNKGGATCTCTCGAG GGAGTTACC (SEQ ID NO:28) was used in combination with a normal downstream primer of sequence 5′-TGGTTCTCTAGAAACTGCTGA (SEQ ID NO:29) to amplify the C-terminus of the CCD and the downstream vector sequences. The 5′ and 3′ ends of the degenerate primer are designed to anneal to the CCD sequence while the degenerate portion (NNKNNKNNKNNKNNKNNK (SEQ ID NO:30)) inserts the adaptein library into the amplicon. For the PCR reaction, plasmid pLPCX DNA (50 ng-20 pg), the 5′ degenerate primer (100 μg), the 3′ primer (100 μg), a 1/10 volume of 10×Taq buffer, dNTP (20 mM), Mg²⁺ (25 mM), and Taq enzyme (5U), are mixed with H₂O to adjust the final volume to 100 μl. The PCR conditions are: 95° C. denaturation for 2 minutes while adding the Taq enzyme, then 30 cycles as follows: 95° C. denaturation for 30 seconds, 59° C. primer annealing for 30 seconds, extension at 72° C. for 30 seconds, with a final extension at 72° C. for 10 minutes. The product of the PCR is analyzed by agarose gel electrophoresis, and the correct band is excised and purified using a standard method (Qiagen kit). The amplicon is analyzed using automated sequencing (Applied Biosystems 377 sequencer and BigDye sequencing kit) and an internal primer to ensure that a mixture of 2 or 4 nucleotides is present at each degenerate site introduced by the degenerate primer. Then the amplicon DNA and plasmid DNA (purified using the Qiagen miniprep kit) are digested overnight at 37° C. with Hind III and Xba I in buffer 2 from Gibco, 1 U/ug of DNA. The amplicon and plasmid DNA are analyzed by agarose gel elecrophoresis and the correct, linear fragments are excised and purified as described above. The amplicon and plasmid DNA are then ligated at a ratio of 1:3 vector:insert, overnight at 14° C. using T4 ligase (Gibco-BRL). The ligation reaction is then cleaned on a spin column (Pharmacia) before being transformed by electroporation in XB1-1 Blue competent E. coli (Stratagene).

Production of Murine Leukemia virus (MLV) library stocks used to deliver adaptein libraries to target cells

Recombinant replication deficient Murine Leukemia viruses (MLV) were used to deliver and express the adaptein library in cells. This virus permits introduction and expression of the adaptein library in a broad range of cell types. The tat-CCD gene was inserted into the packaging vector pLPCX between the BglII and NotI endonuclease restriction sites, or into the packaging vector pLNCX2 between the EcoRI and NotI endonuclease restriction sites. The vector harbors a cDNA copy of an MLV provirus but lacks the structural genes gag, pol and env. What remains is the psi sequence required for efficient packaging of viral RNA and the flanking long terminal repeats (LTR) that function in chromosomal integration and transcription. This vector is bicistronic; the 5′ LTR drives expression of a gene encoding resistance to puromycin and the human cytomegalovirus (CMV) immediate early promoter (P_{CMV IE}) drives adaptein library gene expression.

The adaptein library-containing vector DNA is isolated from E. coli bacteria by standard techniques and purified by equilibrium density gradient separation using cesium chloride to form the gradient and following standard techniques. The DNA (adaptein library) is dissolved at 1 mg/ml in 1 mM EDTA, 10 mM Tris-HCl, pH 8.0 (TE) and stored at −80° C. until required. To produce the MLV encoding the adaptein library, HEK 293 human fibroblasts are grown to 80% confluence on plastic plates in Dulbecco's Modified Eagle Medium (DMEM) supplemented with 10% fetal calf serum (culture medium) and with the bacterial antibiotics streptomycin and penicillin. The cells are simultaneously transfected with vector DNA encoding the adaptein library (25 μg), the MLV structural genes gag-pol (25 μg) and the Vesicular Stomatitis virus glycoprotein (VSV-G; 5 μg). Transfection is conducted according to the methods of Chen and Okayama (Mol. Cell. Biol., 7, 2745-5272 (1987) and used calcium phosphate to form a precipitate with the DNA that is efficiently taken up by this cell type and the encoded genes expressed. The DNA is mixed with 0.1 mM EDTA, 1 mM Tris-HCl, pH 8.0 in purified water to give a total volume of 1.25 ml. To this 1.25 ml of 50 mM BES, 280 mM NaCl, 1.5 mM Na₂HPO₄, pH 7.0 is added and mixed. Calcium chloride (2 M, 0.16 ml) is added dropwise while mixing and then incubated for 10 minutes at room temperature. This is then applied to cells with 2.5 ml of the mixture being used per 15 cm diameter plastic dish containing 25 ml of culture medium. The following day the culture medium is removed and cells washed with 7 ml of fresh medium and replaced with 20 ml of culture medium. To generate sufficient complexity in the library this procedure is repeated 50 times to yield 1×10⁸ transfected cells expressing each library member.

To isolate virus particles containing the adaptein library, the culture supernatants are pooled on the second day after transfection and filtered through a 0.45 μm cellulose acetate filter to remove cells and debris. The MLV-adaptein library-containing medium is then stored at −80° C. in 5 ml aliquots or used immediately. The number of virus particles per ml is enumerated by incubation of HEK 293 cells using serial 10-fold dilutions of the filtered medium and after two days exposing the cells to 1 μg/ml of puromycin. Colonies formed after 5 days gave the initial number of infectious particles. When done using controls (viruses containing the CCD alone), approximately 80% of cells infected at MOI (multiplicity of infection ) of 1.0, and the titers were determined to be greater than >10⁸ plaque forming units per ml.

C. Expression of Adaptein Libraries in Target Cells

The MLV encoded adaptein library is introduced into target cells by seeding cells at 20% confluence on plastic dishes in their required medium and after overnight growth, reaching a confluence of 40% the virus containing culture medium is applied to give a ratio of virus to cells of 1.5:1. This ensures that most target cells received one virus. Two days later the cells are assayed for adaptein library expression or challenged with the pathogenic agent.

### Example 3

### Challenge of Adaptein Library-containing Cells with the Pathogen Rift Valley Fever Virus (RVFV).

RVFV is highly cytopathic in virtually all types of cell cultures that it infects. Initially the live-attenuated vaccine strain MP-12 (Peters and Slone, J. Med. Virol., 10, 45-54 (1982)) will be used. This strain, which is classified as Biosafety Level 2 (BSL-2) and has been well characterized at both the molecular and biological levels, will be used as a model system for pathogenic wild-type strains of RVFV, which is classified at BSL-4. Peripheral inoculation of mice leads to a viremia followed by death from hepataic necrosis (Peters and Anderson, In: Contributions to Epidemiology and Statistics, vol. 3 (Goldblum et al., eds.), S. Karger, Basel, pp. 21-41 (1981); Peters et al., Antiviral Research, 6, 285-297 (1986)). This animal model is a commonly accepted model for human disease, and has been used for many studies with RVFV. Partial protection leads to encephalitis. Inbred rats have been used as models of encephalitis caused by RVFV (Peters and Slone, J. Med. Virol., 10, 45-54 (1982)). The assays described herein to identify adapteins that protect cells and animals from challenge with RVFV can be modified for use with different pathogens and different cells using methods known to the art.

Vero cells, which are uniformly susceptible to cytolytic infection with RVFV, are in challenge experiments. Vero cells expressing an adaptein library are challenged at a multiplicity of infection (MOI) of 0.1. After incubation at 37° C. for 48 hours (or 35° C. for MP-12), 100% of cells normally show cytopathic effects (CPE). However, cells expressing a protective adaptein survive in the presence of viral replication in surrounding cells and repeated challenge from virus in the cell culture supernatant.

Cells expressing an adaptein that protects against RVFV challenge survives and continues replicating, producing small cell colonies on the plastic surface after CPE is complete in surrounding, unprotected cells. PCR- or cloning-based protocols are used to identify the protective adaptein sequence from each of these colonies. If evidence of a mixed adaptein population is found, (for instance, two or more sequences), several DNA clones of a PCR amplicon will be sequenced to assess the sequence diversity in the population. Protective adapteins are recloned into retroviral vectors, and cells infected with these single-adaptein retroviruses are challenged with RVFV to ensure that most to all infected cells are protected. Protection is assessed by continued cell proliferation, reduction or lack of infectious virus in the culture supernatant, and lack of detectable viral RNA in cells compared to control virus-infected cultures.

After confirming the efficacy of retrovirus containing a single protective adaptein, other cell types (e.g., hepatocyte, neuronal, primary human macrophages and monocytic cell lines) are tested to determine if protection is cell-type specific. Cell protection after challenge is assayed by decreased viral replication compared to negative controls, reduction or lack of infectious virus in the culture supernatant, and delayed or reduced CPE.

The initial RVFV selection procedures in cell culture may overwhelm cells with the highly virulent RVFV. If no resistant cells are detected, the stringency of the challenge can be modified to increase the likelihood of cell survival, including: reducing the MOI of the initial RVFV challenge to delay viral replication; using MP-12 at partially non-permissive temperatures, e.g., 37-39° C; periodically replacing the cell culture medium to prevent continuous virus challenge of the surviving cells; or adding neutralizing antibody to the supernatant to reduce continuous challenge of the surviving cells. Alternatively, other less stringent Bunyaviridae viruses, such as Punta Toro or LaCrosse, can be used for initial implementation.

### Example 4

### Evaluate Efficacy of Recombinant Anti-RVFV Adapteins in Cell Culture and Animal Models

Protective adapteins are subcloned into bacterial expression vectors, expressed, and purified. Minimum adaptein toxicity levels will be established using standard protocols. Adaptein entry into cultured cells is confirmed by immunofluorescence microscopy of Tat-CCD as described above. Cytoplasmic immunofluorescence indicates that the agent has reached the desired compartment. To assess the distribution of each protective adaptein in vivo, 0.1, 1, 10 and 100 nanomoles (nmol) of it is inoculated intraperitoneally into mice, and at 2, 4, 12, 24 and 38 hours, the brain, liver, heart muscle, lung, spleen and peritoneal lymph nodes are dissected. Confocal fluorescence microscopy of deparaffnized sections are used to assess the targeting of the adapteins to appropriate sites, especially the endothelial and hepatic tissues (believed to determine the course of RVF pathogenesis) and the brain (where infection is associated with encephalitis).

Next, the ability of cell-permeant protective adapteins to shield cultured cells and animals against RVFV challenge is tested. Cells will be pretreated with a range of subtoxic adaptein concentrations before challenge with RVFV. CPE is monitored and compared with controls; viral replication is assessed by determining the infectious titers in the supernatant after various incubation times (12, 24, 36, 48 hours). Adapteins showing efficacy in cell culture are tested in NIH Swiss mice treated with cell-permeant adapteins at subtoxic intraperitoneal and intravenous doses before virus challenge; aerosol challenge is performed on compounds showing initial efficacy. Based on previous studies with Tat-β-Gal (Schwarze et al., Science, 285, 1569-1572 (1999), it is expected that doses in the range of 1-500 μg will be administered initially, with lethality assessed using 5 animals per group. Adapteins with antiviral activity are then retested in additional mice and full pathological studies of these animals performed. These include complete necropsies with conventional histological techniques to assess tissue pathology, determination of viral load in various organs such as lymph nodes and brain by plaque assays, and immunohistology for localization of viral antigen in various organs. Viremia is monitored and average survival times of virus-infected animals is compared. Post-exposure efficacy and combinations of partially protective adapteins are also evaluated.

### Example 5

### Evaluate Shared Protective Action of Anti-RVFV Adapteins by Challenging Cell and Animal Models with Diverse Bunyaviruses

Adapteins that protect cells against challenge by RVFV are tested against members of all four genera of Bunyaviridae: Bunyavirus (LaCrosse, Bunyamwera), Nairovirus (Crimean Congo HF), Phlebovirus (RVF plus Punta Toro, Sandfly Sicilian), and Hantavirus (Hantaan, Sin Nombre). Testing of these agents is done in cell culture; animal tests, similar to those described above, are undertaken.

### Example 6

### Recombinant Carrier Protein Crosses Cell Membranes and Accumulates Within the Cytoplasm

We examined the ability of purified recombinant Tat-CCD carrier protein to cross cell membranes. Recombinant tat-CCD was isolated from BL21(DE3) cells grown at 37° C., induced with 1 mM isopropyl-thio-β-D-galactoside (IPTG) for 2-4 hours and recovered from the supernatant after mild sonication of bacterial pellets in ice-cold lysis buffer (20 mM Tris-HCl pH 7.3, 5 mM DTT, 150 mM NaCl, 5% glycerol, 2 mM EDTA). The tat-CCD was purified using cation exchange HPLC (Poros 20 SP column, Perseptive Biosystems) in chilled MOPS buffer (20 mM MOPS pH 7.3, 5 mM DTT) and size exclusion chromatography (Biosep SEC-S3000, Phenomenex) in chilled Tris buffer (10 mM Tris pH 7.3, 150 mM NaCl, 10 mM DTT). Purified tat-CCD was concentrated to 30 mg/ml using ultrafiltration (Amicon).

Polyclonal antibody to CCD was made by injecting rabbits with CCD in Freund's adjuvant.

Cultured Vero cells were incubated with 1 μM Tat-CCD protein for 20 minutes at 37° C., then extensively washed in PBS buffer to remove extracellular protein. Cells were fixed in glutaldehyde, permealized with Triton X-100, and incubated with anti-CCD polyclonal antibody, and then with FITC-conjugated anti-rabbit secondary antibody. Cells were examined by immunofluorescence microscopy. Tat-CCD clearly localized to the cytoplasm of Vero cells. No nuclear localization was observed for Tat-CCD. In addition, no immunofluorescence was observed in control cells incubated with similar concentrations of recombinant CCD protein.

The complete disclosure of all patents, patent applications, and publications, and electronically available material (e.g., GenBank amino acid and nucleotide sequence submissions, and computer programs) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

### Sequence Listing Free Text

| | |
|---|---|
| SEQ ID NO: 2 | Cell-permeant polypeptide |
| SEQ ID NO: 3 | Cell-permeant polypeptide |
| SEQ ID NO: 4 | Cell-permeant polypeptide |
| SEQ ID NO: 5 | Cell-permeant polypeptide |
| SEQ ID NO: 6 | Nucleotide sequence encoding tat-CCD |
| SEQ ID NO: 7 | Amino acid sequence of tat-CCD |
| SEQ ID NO: 9-10 | Primer |
| SEQ ID NO: 11 | A variable region amino acid sequence |
| SEQ ID NO: 13-30 | Primer |
| SEQ ID NO: 31 | Adaptein-1 nucleotide sequence |
| SEQ ID NO: 32 | Adaptein-2 nucleotide sequence |
| SEQ ID NO: 33 | Adaptein-1 amino acid sequence |
| SEQ ID NO: 34 | Adaptein-2 amino acid sequence |

What is claimed is:

1. A collection of polypeptides comprising at least two polypeptides, each polypeptide comprising a fragment of SEQ ID NO:1 beginning at any amino acid from about 119 to about 124 and ending at any amino acid from about 258 to about 275, wherein at least two consecutive amino acids within the regions of amino acids 129-137, or amino acids 182-189, or amino acids 257-264 as depicted at SEQ ID NO:1 are replaced by an amino acid sequence comprising Xaaₙ, wherein n is from about 5 to about 21, and each Xaa is independently a random amino acid.

2. The collection of claim 1 wherein each member of the collection further comprises a cell-permeant region fused to the amino terminal end of the polypeptide.

3. The collection of claim 2 wherein the cell-permeant region comprises an amino acid sequence selected from the group consisting of YGRKKRRQRRR (SEQ ID NO:2), RQIKIWFQNRRMKWKK (SEQ ID NO:3), RQIKIWFPNRRMKWKK (SEQ ID NO:4), and RQPKIWFPNRRPKWKK (SEQ ID NO:5).

4. The collection of claim 1 wherein the collection comprises a polypeptide selected from the group consisting of SEQ ID NO:33 and SEQ ID NO:34.

5. A cell comprising a member of the collection of claim 1.

6. A population of cells comprising two or more cells, wherein each member of the population comprises one polypeptide of the collection of claim 1.

7. A collection of polypeptides wherein the collection comprises a polypeptide selected from the group consisting of SEQ ID NO:33 and SEQ ID NO:34.

8. A polypeptide selected from the group consisting of
an amino acid sequence SEQ ID NO:2 fused to an amino terminal end of a fragment of SEQ ID NO:1 beginning at any amino acid from about 119 to about 124 and ending at any amino acid from about 258 to about 275, wherein at least two consecutive amino acids within the regions of amino acids 129-137, or amino acids 182-189, or amino acids 257-264 as depicted at SEQ ID NO:1 are replaced by an amino acid sequence comprising Xaaₙ, wherein n is from about 5 to about 21, and each Xaa is independently a random amino acid.

9. The polypeptide of claim 8 wherein the polypeptide further comprises a cell-permeant region fused to the amino terminal end of the polypeptide.

10. The polypeptide of claim 9 wherein the cell-permeant region comprises an amino acid sequence selected from the group consisting of YGRKKRRQRRR (SEQ ID NO:2), RQIKIWFQNRRMKWKK (SEQ ID NO:3), RQIKIWFPNRRMKWKK (SEQ ID NO:4), and RQPKIWFPNRRPKWKK (SEQ ID NO:5).

11. A cell comprising the polypeptide of claim 8.

12. A collection of polynucleotides comprising at least two polynucleotides, each polynucleotide comprising a coding sequence encoding a polypeptide comprising a fragment of SEQ ID NO:1 beginning at any amino acid from about 119 to about 124 and ending at any amino acid from about 262 to about 275, wherein at least two consecutive amino acids within the regions of amino acids 129-137, or amino acids 182-189, or amino acids 257-264 as depicted at SEQ ID NO:1 are replaced by an amino acid sequence comprising Xaaₙ, wherein n is from about 5 to about 21, and each Xaa is independently a random amino acid.

13. The collection of claim 12 wherein the polypeptide encoded by the coding sequence of each member of the collection further comprises a cell-permeant region fused to the amino terminal end of the polypeptide.

14. The collection of claim 13 wherein the cell-permeant region comprises an amino acid sequence selected from the group consisting of YGRKKRRQRRR (SEQ ID NO:2), RQIKIWFQNRRMKWKK (SEQ ID NO:3), RQIKIWFPNRRMKWKK (SEQ ID NO:4), and RQPKIWFPNRRPKWKK (SEQ ID NO:5).

15. The collection of claim 14 wherein the nucleotide sequence of the coding sequence encoding the Xaaₙ consists of a nucleotide sequence NNKₘ, wherein N is independently a random nucleotide, K is independently a guanine or a thymine, and wherein o is from about 5 to about 21.

16. A vector comprising a member of the collection of claim 12.

17. The collection of claim 16 wherein the vector is a retrovirus.

18. A cell comprising a member of the collection of claim 12.

19. A population of cells comprising two or more cells, wherein each member of the population comprises one polynucleotide of the collection of claim 12.

20. A method for crystallizing a polypeptide comprising an amino acid sequence SEQ ID NO:1, the method comprising:
preparing purified polypeptide comprising an amino acid sequence SEQ ID NO:1 at a concentration of about 3 mg/ml to about 20 mg/ml; and
crystallizing the polypeptide comprising an amino acid sequence SEQ ID NO:1 from a solution comprising about 20% by weight to about 28% by weight polyethylene glycol, about 0.05 M to about 0.2 M ammonium sulfate, and about 1 mM to about 20 mM urea, wherein the solution is buffered to a pH of about 6 to about 8.

21. A method for crystallizing a polypeptide comprising an amino acid sequence SEQ ID NO:1, the method comprising:
preparing purified polypeptide comprising an amino acid sequence SEQ ID NO:1 at a concentration of about 3 mg/ml to about 20 mg/ml; and
crystallizing the polypeptide comprising an amino acid sequence SEQ ID NO:1 from a solution comprising about 15% by weight to about 25% by weight polyethylene glycol 4000, and about 0.05 M to about 0.4 M MgCl₂, wherein the solution is buffered to a pH of about 6 to about 8.

22. A crystal of a polypeptide comprising an amino acid sequence SEQ ID NO:1.

23. The crystal of claim 22 having the space group symmetry P2₁2₁2₁.

24. The crystal of claim 22 comprising a unit cell having dimensions of a, b, and c; wherein a is about 69.3 Å to about 72.0 Å, b is about 75.2 Å to about 76.0 Å, and c is about 90.1 Å to about 94.7 Å; and wherein α=β=γ=about 90°.

25. A method for identifying a polypeptide within a collection that prevents cell death after exposure to a pathogen or a toxin, the method comprising:
providing a cell comprising a polypeptide that is a member of a collection of polypeptides comprising at least two polypeptides, each polypeptide comprising fragment of SEQ ID NO:1 beginning at any amino acid from about 119 to about 124 and ending at any amino acid from about 258 to about 275, wherein at least two consecutive amino acids within the regions of amino acids 129-137, or amino acids 182-189, or amino acids 257-264 as depicted at SEQ ID NO:1 are replaced by an amino acid sequence comprising Xaaₙ, wherein n is from about 5 to about 21, and each Xaa is independently a random amino acid.;
exposing the cell to a pathogen or a toxin; and
determining whether the polypeptide prevents cell death, comprising:
incubating the cell under conditions such that the pathogen or the toxin kills a cell that does not comprise a polypeptide that prevents cell death after exposure to a pathogen or a toxin, wherein the presence of a cell that proliferates indicates the polypeptide prevents cell death after exposure to a pathogen or a toxin.

26. The method of claim 25 wherein the pathogen is selected from the group consisting of a virus and a microbe.

27. The method of claim 26 wherein the microbe is selected from the group consisting of a bacterium, a rickettsia, and a fungus.

28. The method of claim 25 wherein the toxin is a biological toxin.

29. The method of claim 25 wherein the toxin is a chemical toxin.

30. A method for identifying a polypeptide within a collection that binds a pathogen, a toxin, a polypeptide, or a polynucleotide, the method comprising:
providing a cell comprising a polypeptide that is a member of a collection of polypeptides comprising at least two polypeptides, each polypeptide comprising fragment of SEQ ID NO:1 beginning at any amino acid from about 119 to about 124 and ending at any amino acid from about 258 to about 275, wherein at least two consecutive amino acids within the regions of amino acids 129-137, or amino acids 182-189, or amino acids 257-264 as depicted at SEQ ID NO:1 are replaced by an amino acid sequence comprising Xaaₙ, wherein n is from about 5 to about 21, and each Xaa is independently a random amino acid;
exposing the cell to a pathogen or a toxin; and
determining whether the polypeptide binds the pathogen or the toxin, comprising:
incubating the cell under conditions such that the pathogen or the toxin kills a cell that does not comprise the polypeptide, wherein the presence of a cell that proliferates indicates the polypeptide binds the pathogen, the toxin, a polypeptide, or a polynucleotide.

31. The method of claim 30 wherein the pathogen is selected from the group consisting of a virus and a microbe.

32. The method of claim 31 wherein the microbe is selected from the group consisting of a bacterium, a rickettsia, and a fungus.

33. The method of claim 30 wherein the agent is a biological toxin.

34. The method of claim 30 wherein the agent is a chemical toxin.
